(19) United States
(12) Patent Application Publication
(10) Pub. No.: US 2017/0164898 A1
(43) Pub. Date: Jun. 15, 2017

(54) Implantable Electrode Line and Set of Electrode Line Modules

(71) Applicant: BIOTRONIK SE & Co. KG, Berlin (DE)

(72) Inventors: Dajana Kaiser, Berlin (DE); Detmar Jadwizak, Erkner (DE); Carsten Fruendt, Berlin (DE); Gordon Hillebrand, Berlin (DE)

(21) Appl. No.: 15/359,831

(22) Filed: Nov. 23, 2016

(30) Foreign Application Priority Data
Dec. 15, 2015 (DE) 10 2015 121 815.1

(51) Int. Cl. A61B 5/00; A61N 1/39; A61N 1/365

(52) U.S. Cl. CPC A61B 5/686; A61N 1/365; A61N 1/3956; A61N 1/3987

(57) ABSTRACT

Implantable electrode line electrically stimulating and/or sensing bodily tissue, including a distal end portion having at least one electrode pole and/or sensor, a proximal end portion having at least one device connection contact, and a line body containing an electrical feed line connecting the electrode pole and/or sensor to the device connection contact. The distal end portion, proximal end portion, and line body are separate modules. The distal end portion module has, at its proximal end, at least one first connection contact. The proximal end portion module has, at its distal end, at least one second connection contact. The line body module has, at its distal end, a third connection contact for detachable engagement with the first connection contact and, at its proximal end, a fourth connection contact for detachable engagement with the second connection contact. The modules form the electrode line in the connected state of the connection contacts.

### CROSS-REFERENCE TO RELATED APPLICATIONS

This patent application claims the benefit of and priority to co-pending German Patent Application No. DE 10 2015 121 815.1, filed on Dec. 15, 2015 in the German Patent Office, which is hereby incorporated by reference in its entirety.

### FIELD OF THE INVENTION

The present invention relates to an implantable electrode line for electrically stimulating and/or for sensing bodily tissue, comprising a distal end portion which has at least one electrode pole and/or a sensor, a proximal end portion which has at least one device connection contact, and a line body which contains an electrical feed line for connecting the electrode pole and/or sensor to the device connection contact. The present invention also relates to a set of electrical line modules for forming an electrode line of this type.

### BACKGROUND

Implantable electrode lines of the above-mentioned type have been known for decades and used on a mass scale, in particular, in conjunction with cardiac pacemakers or implantable defibrillators, but also with other medical electronic implants, such as, for example, neurostimulators. There are a wide variety of electrode lines of this type, which are adapted to various uses and anatomical environments of the patients to be treated (who, in addition to adults, can also be children and, in particular, cases can also be other mammals).

This multiplicity of electrode lines has, in the meantime, become difficult to manage for the user and leads to problems when it comes to production and storage, and in particular cases means that an electrode line is used which, although immediately available, is not best suited for the specific purpose. It goes without saying that the cost situation with such a large array of products having a similar purpose also might not be optimal.

A modular line connector for implantable lines with which the proximal or distal end of a provided electrode line can be extended by a conductive element is known from European Patent No. 2 281 327 B1. U.S. Publication No. 2014/0316502A1 discloses an electrode line having a modularly constructed line end with which a multiplicity of conductors can be connected to the feed lines in the line body.

The present invention is directed toward overcoming one or more of the above-mentioned problems.

### SUMMARY

The invention above addresses the problem of specifying an implantable electrode line of this type which can be adapted to various uses and anatomical environments and therefore contributes to reducing the effort associated with the production, logistics and storage of many different electrode lines and also contributes to lowering the resultant costs.

At least this problem is solved by an implantable electrode line having the features of claim 1. Expedient developments of the inventive concept are disclosed in the dependent claims. A set of electrode line modules for forming the proposed electrode line is also disclosed.

The present invention includes the consideration of constructing an implantable electrode line in a modular manner on the whole, more specifically from the proximal device connection to the distal line tip. More specifically, a construction from three basic modules is proposed, that is to say a module forming the distal end portion, a module forming the proximal end portion, and a module forming the line body between both end portions.

More specifically, the present invention teaches that the distal end portion, proximal end portion, and line body are separate modules, wherein the distal end portion module has, at its proximal end, at least one first connection contact; the proximal end portion module has, at its distal end, at least one second connection contact; and the line body module has, at its distal end, a third connection contact designed for detachable engagement with the first connection contact and has, at its proximal end, a fourth connection contact designed for detachable engagement with the second connection contact, and the modules form the electrode line in the connected state of the connection contacts.

The proposed solution makes it possible to form implantable electrode lines for a wide field of application and for practically all anatomical environments from a manageable set of modules for the specified main portions of the electrode line. The production of a very large number of different types of complete electrode lines can thus be avoided, which can reduce the overall costs of providing medical electrode lines. Furthermore, the production and storage of implantable electrode lines is simplified on the whole, which can lead to further cost savings. Lastly, the provision of an electrode line optimally adapted to the particular use and the given anatomy is facilitated and in certain situations is actually made possible for the first time, which can lead in principle to an increase in the quality of patient care.

In one embodiment of the present invention, the distal end portion module and/or the proximal end portion module and/or the line body module comprise/comprises a number of sub-modules. A further improved adaptation of the electrode line according to the present invention to the numerous possible therapy and diagnosis methods with use of modern electro-medical implants is ensured as a result.

In one embodiment, the line body module comprises a plurality of sub-modules which can be connected in series and which, in particular, each have, at one end, at least one connection contact of the first connection contact type and, at the other end, a connection contact of the third connection contact type. More specifically, provision is made here so that the line body module comprises at least one sub-module which has an electrode pole, in particular, a ring electrode, or a shock coil. As a result of this embodiment, a stimulation or defibrillation portion can also be incorporated in the middle region of an electrode line, without the need to design, manufacture and store a specific complete electrode line for this relatively rarely required configuration.

In a further embodiment of the present invention, the distal end portion module comprises a plurality of sub-modules which can be connected in series and all of which, with the exception of the sub-module forming the distal end (the tip) of the electrode line, have, in particular, at one end, a connection contact of the third connection contact type and, at another end, a connection contact of the first connection contact type. Various stimulation configurations or stimulation and sensing configurations can thus be provided in a very wide range of applications in a simple and economical manner.

In a further embodiment, the proximal end portion module comprises a plurality of sub-modules each having at least one device connection contact and one connection contact of the second connection contact type, and also comprises a branching module, which carries the second connection contact and has a plurality of connection contacts of the fourth connection contact type. The proposed electrode line can thus be adapted easily and in an uncomplicated manner to different stimulation configurations and other device configurations, especially also to configurations which comprise a plurality of signal sources and/or signal detection devices.

Whereas the intervention can already be provided in a conductor/electrode pole configuration having a single electrode pole and a single feed line, an embodiment as a “multi-core” electrode line is currently of greater practical significance. In such an embodiment, the distal end portion module comprises a multiplicity of first connection contacts, the proximal end portion module has a multiplicity of second connection contacts, and the line body module has a multiplicity of third connection contacts and a multiplicity of fourth connection contacts, and the number of connection contacts of all types is the same. In an exemplary configuration, four feed lines are provided in the electrode line, and the number of all connection contacts is in each case 4 accordingly. It goes without saying that the electrode line can also have a different number of feed lines and corresponding connection contacts of the specified types.

In a further embodiment, the line body module comprises a multiplicity of conductors, to which the connection contact or each third and fourth connection contact is/are attached by crimping or laser welding. In principle, the configuring of the final electrode line configuration is separated here from the production process of the line body and is even possible immediately before implantation. In accordance with one embodiment, provision is made so that only some of the conductors in the line body module are provided with a third and fourth connection contact. The remaining conductors then remain without electrical connection and therefore without function.

In an embodiment that is advantageous in terms of application, the first connection contacts and third connection contacts engaging herein are formed as plug/socket contact pairs. The modules can thus be easily electrically and mechanically connected to one another. In one embodiment, a locking of the modules in order to prevent accidental detachment from one another is additionally provided, wherein the first and third connection contacts and also second and fourth connection contacts are designed especially for reciprocal mechanical locking in the form of a bayonet fitting. However, within the scope of the present invention, other mechanical fixing or locking means can also be used, including those which do not act between the individual connection contacts, but instead between the modules as a whole.

In a further expedient embodiment, the proximal end portion and line body modules have a continuous central lumen for a guide wire or mandrel. Part of the longitudinal extension of the distal end portion module will typically also have a lumen of this type so that the guide wire or mandrel used during the insertion process can also penetrate the distal end portion module a little and the tip of the electrode line can thus also be selectively guided.

In one embodiment of the set of electrode line modules according to the present invention, this comprises a plurality of line body modules of different lengths and/or alternatively proximal end portion modules which can be connected alternatively to the line body module and which have different device connection contacts and/or a plurality of distal end portions which can be connected alternatively to the line body module and which have different embodiments of electrode poles and/or a different mechanical/geometrical embodiment. With such a module set, practically all relevant fields of use of the electrode line can be covered in a modular manner depending on the scope of the embodiment of said set.

Further embodiments, features, aspects, objects, advantages, and possible applications of the present invention could be learned from the following description, in combination with the Figures, and the appended claims.

### DESCRIPTION OF THE DRAWINGS

Advantages and expedient features of the present invention will become clear incidentally from the following description of an exemplary embodiment with reference to the drawings, in which:

FIG. 1 shows an overall view of an implantable electrode line, connected to a cardiac pacemaker,

FIG. 2 shows three different proximal end portion modules of an exemplary embodiment of the electrode line according to the present invention,

FIG. 3 shows two line body modules of an exemplary embodiment of the electrode line according to the present invention,

FIG. 4 shows various sub-modules for forming a line body module of an exemplary embodiment of the electrode line according to the present invention,

FIG. 5 shows three distal end portion modules of an exemplary embodiment of the electrode line according to the present invention,

FIG. 6 shows a schematic illustration to explain the conductor configuration and connection contact configuration at a module connection point of an exemplary embodiment of the electrode line according to the present invention,

FIG. 7 shows perspective schematic illustrations of an embodiment of the first and third, and second and fourth connection contacts of an exemplary embodiment of the electrode line according to the present invention, and

FIG. 8 shows a schematic longitudinal sectional illustration of a connection contact region of an exemplary embodiment of the electrode line according to the present invention.

### DETAILED DESCRIPTION

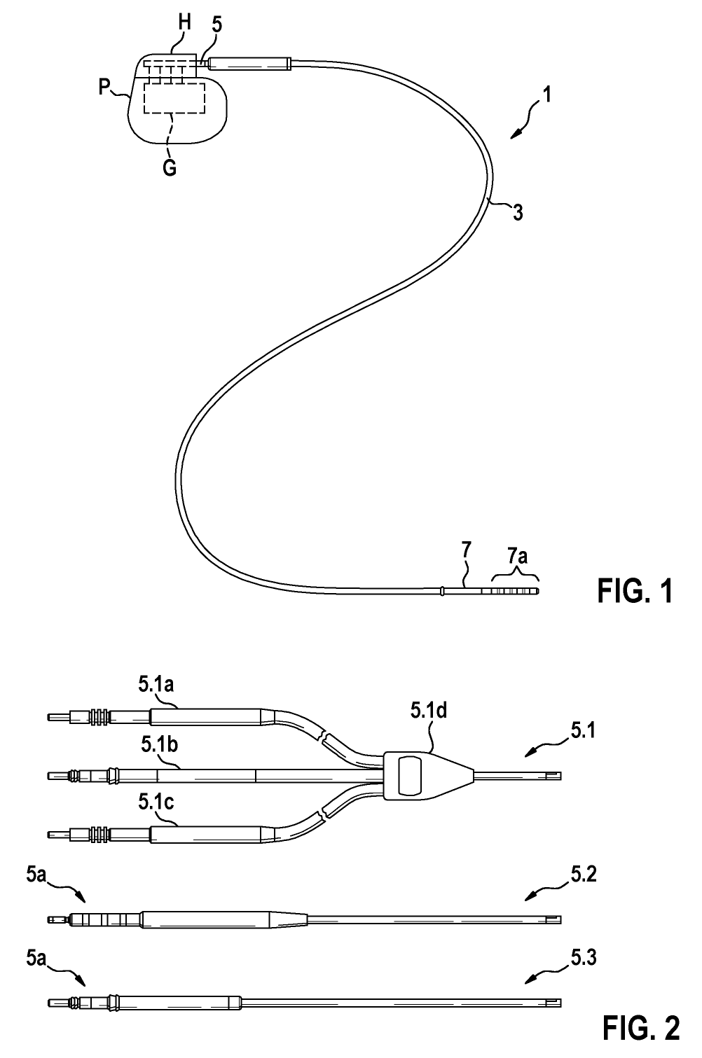

FIG. 1 shows an overall view of a modularly constructed electrode line 1, which comprises a line body module 3, a proximal end portion (plug) module 5, and a distal end portion (electrode pole) module 7. The electrode line 1 is connected to the header H of a cardiac pacemaker P via the proximal end portion module 5. The electrode line delivers stimulation pulses, which are generated in a pulse generator F of the cardiac pacemaker P, to the heart (not shown) of a patient fitted with a pacemaker via a plurality of electrode poles 7a in the distal end portion module 7.

FIG. 2 shows three various proximal end portion modules 5.1, 5.2 and 5.3 for forming various embodiments of an electrode line 1 of the type shown in FIG. 1 for various purposes. Whereas the proximal end portion modules 5.2 and 5.3 are each formed in one piece and differ merely in terms of the specific embodiment of an individual connection plug 5a, the module 5.1 consists of three sub-modules 5.1a, 5.1b and 5.1c, by means of which the electrode line can be connected in parallel to three different connection sockets of a stimulation device, and a branching module 5.1d, which brings together the lines of the three sub-modules 5.1a, 5.1b, and 5.1c and forms a conductor configuration which can be attached to the line body module (not shown here).

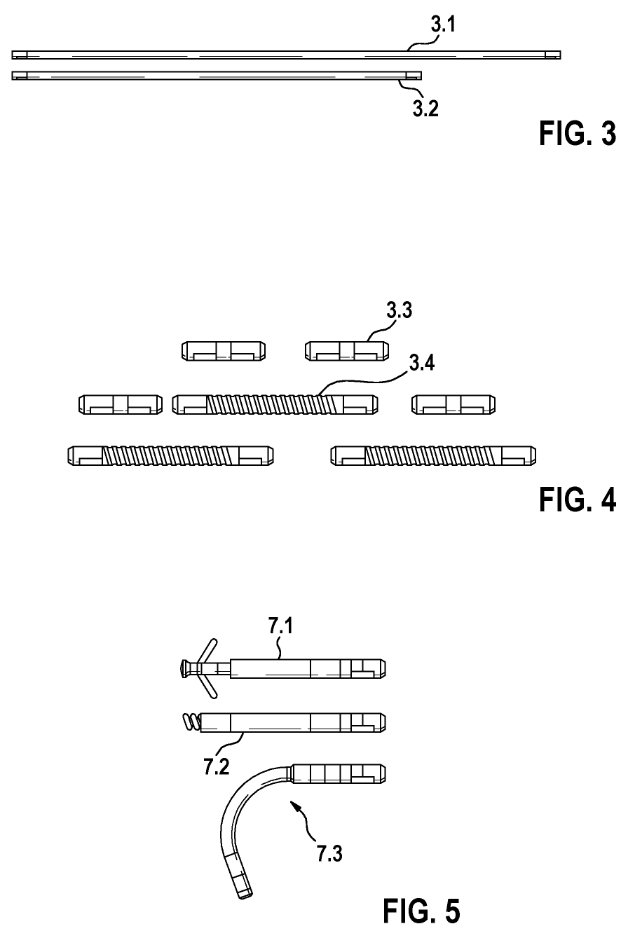

FIG. 3 shows two different line body modules 3.1 and 3.2 of a set of electrode line modules for forming various embodiments of an electrode line 1 of the type shown in FIG. 1. The modules 3.1 and 3.2 are identical in terms of their structure and differ merely in terms of their length.

FIG. 4 shows various sub-modules 3.3 and 3.4, which can be used to form a line body module of more complex structure. Here, the sub-modules are a ring electrode sub-module 3.3 for stimulation purposes and a shock coil sub-module 3.4 for an electrode line that can be attached to a cardioverter and that can be used therewith. It goes without saying that these sub-modules can be used in conjunction with further sub-modules with which an elongate line body of the electrode line can be formed. These could be modules or sub-modules of the type shown in FIG. 3.

FIG. 5 shows three various distal end portion modules 7.1, 7.2 and 7.3, which can be connected alternatively to a line body module in order to form different stimulation configurations. Whereas the sub-modules 7.1 and 7.2 differ in terms of the means for anchoring the electrode line tip at the bodily tissue (heart tissue) to be stimulated, specifically with the presence of a barbed tip (module 7.1) or a screw-in tip (module 7.2), the module 7.3 is J-shaped and also differs from the two other modules in terms of its electrode pole configuration. Since the specific embodiment of fixing means and electrode configurations of a distal end portion module is not of vital importance in conjunction with the present invention, there is no need to provide a more detailed description of these modules here.

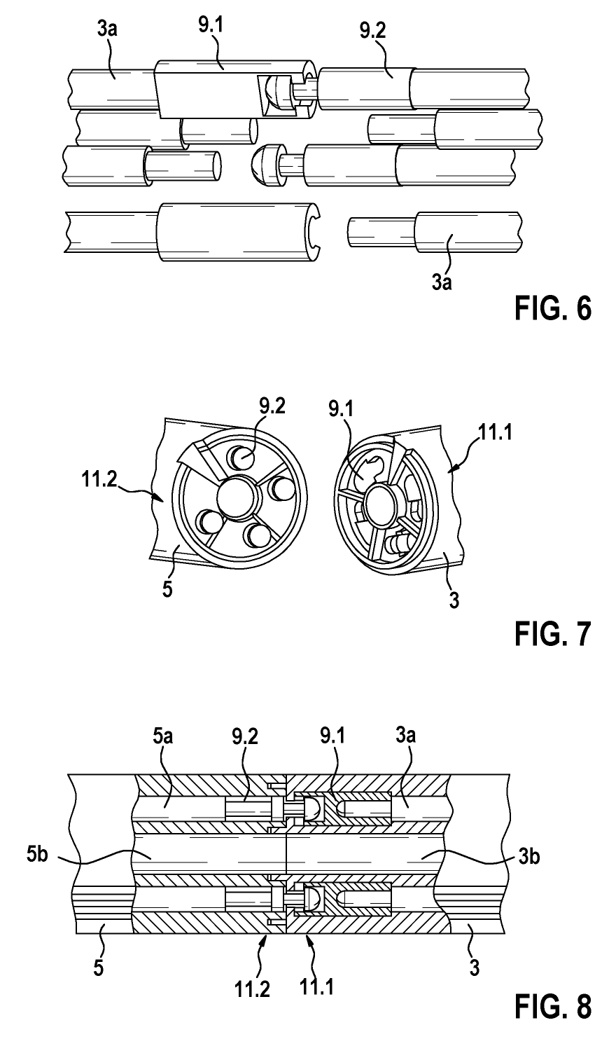

FIG. 6 schematically shows an exemplary conductor structure of an electrode line constructed in a modular manner in accordance with the present invention, which conductor structure comprises four feed lines (conductors) 3a. The conductors 3a individually insulated and additionally embedded in an insulating line body (not shown) are provided at a separation point between different modules or sub-modules with connection contacts 9.1, 9.2 in the form of a plug-socket connection. In the drawing, one of the feed lines is illustrated in a state in which the two conductors 3a abutting one another in the longitudinal direction are (detachably) connected to one another by means of connection contacts 9.1 and 9.2 fastened thereto. The conductors of a second possible line connection are shown with the connection contacts 9.1 and 9.2 attached thereto, but still in the unconnected state, and the remaining conductors are illustrated with free connection ends, but without attached connection contacts. This is thus a synoptic schematic illustration, which does not show the state of use of a finished electrode line. However, it is conceivable that for certain purposes, even in the case of finished electrode lines, not all embedded conductors will be used as effective electrical connections, and therefore will not be provided with connection contacts.

FIG. 7 shows, in a schematic perspective illustration, the end faces of two modules of an electrode line according to the present invention—denoted here by way of example with the numbers 3 for a line body module and 5 for a proximal end portion module. Similarly to the embodiment illustrated schematically in FIG. 6 with two feed lines, both modules 4, 5 each have four connection contacts 9.1 and 9.2 respectively, which protrude from the end face of the corresponding module (connection contacts 9.2 in the case of the module 5) or are incorporated therein (connection contacts 9.1 in the module 3).

It can also be seen that the module end faces have a specific locking and sealing structure 11.1 and 11.2, the function of which is based on a bayonet fitting. These locking and sealing structures 11.1, 11.2 enable, in practice, a very comfortable connection of the adjacently arranged modules by gripping by hand, and nevertheless ensure a reliable and tight electrical and mechanical connection.

FIG. 8 shows the corresponding structure in greater detail in a longitudinal sectional illustration. Here, the conductors of the line body module 3, as in FIG. 6, are denoted by the number 3a, whereas feed lines in the proximal end portion module 5 connected thereto are denoted by the number 5a. The structure of the locking and sealing structures 11.1, 11.2, which comprise silicone sealing lips and “chicanes” (not denoted separately), can be seen here in greater detail. It can also be seen that the modules 3, 5 each have a central lumen 3b and 5b, respectively, and these lumina are aligned longitudinally with one another when the modules are connected, such that an accordingly constructive electrode line can be implanted in the usual way with a guide wire or mandrel.

With reference to FIGS. 6 to 8 on the one hand and the accompanying claims and also the above general embodiments on the other hand, the following should be noted: The drawings show two types of connection contacts by way of example, specifically, a “female” contact 9.1 and a “male” contact 9.2, with which a plug connection in the form of a plug-socket connection is formed.

The first to fourth connection contacts mentioned further above and in the claims can be provided by means of these two types of connection contacts. The terms “first”, “second”, “third” and “fourth” connection contacts mean that, at the ends of the corresponding modules, connection contacts of a certain type are provided, to which connection contacts of another type on an adjacent module will fit (detachably). Since, in the case of the electrode line according to the present invention, three modules are connected in series, two physically different types of contacts are sufficient to provide the first to fourth connection contacts; however, it is also possible to carry out the invention actually with four physically different contacts.

In addition, the present invention can also be carried out in a large number of modifications of the examples shown here and aspects of the invention detailed further above.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/We claim:

1. An implantable electrode line for electrically stimulating and/or for sensing bodily tissue, comprising:
a distal end portion which has at least one electrode pole and/or a sensor;
a proximal end portion which has at least one device connection contact; and
a line body which contains an electrical feed line for connecting the electrode pole and/or sensor to the device connection contact,
wherein the distal end portion, proximal end portion, and line body are separate modules, wherein the distal end portion module has, at its proximal end, at least one first connection contact, the proximal end portion module has, at its distal end, at least one second connection contact, and the line body module has, at its distal end, a third connection contact designed for detachable engagement with the first connection contact and, at its proximal end, a fourth connection contact designed for detachable engagement with the second connection contact, and the modules form the electrode line in the connected state of the connection contacts.

2. The implantable electrode line according to claim 1, wherein the distal end portion module and/or the proximal end portion module and/or the line body module comprise/comprises a number of sub-modules.

3. The implantable electrode line according to claim 2, wherein the line body module comprises a plurality of sub-modules which can be connected in series and which in particular each have, at one end, at least one connection contact of the first connection contact type and, at the other end, a connection contact of the third connection contact type.

4. The implantable electrode line according to claim 3, wherein the line body module comprises at least one sub-module which has an electrode pole, in particular a ring electrode, or a shock coil.

5. The implantable electrode line according to claim 2, wherein the distal end portion module comprises a plurality of sub-modules which can be connected in series and all of which, with the exception of the sub-module forming the distal end of the electrode line, have, in particular at one end, a connection contact of the third connection contact type and, at another end, a connection contact of the first connection contact type.

6. The implantable electrode line according to claim 2, wherein the proximal end portion module comprises a plurality of sub-modules each having at least one device connection contact and one connection contact of the second connection contact type, and also comprises a branching module, which carries the second connection contact and in particular has a plurality of connection contacts of the fourth connection contact type.

7. The implantable electrode line according to claim 1, wherein the distal end portion module has a multiplicity of first connection contacts, the proximal end portion module has a multiplicity of second connection contacts, and the line body module has a multiplicity of third connection contacts and a multiplicity of fourth connection contacts, and the number of connection contacts of all types is the same.

8. The implantable electrode line according to claim 1, wherein the line body module comprises a multiplicity of conductors, to which the connection contact or each third and fourth connection contact is/are attached by crimping or laser welding.

9. The implantable electrode line according to claim 8, wherein, in the line body module, only some of the conductors are provided with a third and fourth connection contact.

10. The implantable electrode line according to claim 1, wherein the first and third connection contacts, said third connection contacts engaging in said first connection contacts, and also the second and fourth connection contacts, said fourth connection contacts engaging in said second connection contacts, are formed as plug/socket contact pairs.

11. The implantable electrode line according to claim 10, wherein the first and third connection contacts and also second and fourth connection contacts are designed for reciprocal mechanical locking in the form of a bayonet fitting.

12. The implantable electrode line according to claim 1, wherein the proximal end portion and line body modules have a continuous central lumen for a guide wire or mandrel.

13. A set of electrode line modules for forming an implantable electrode line according to claim 1, comprising at least one distal end portion module, at least one proximal end portion module, and at least one line body module.

14. The set of electrode line modules according to claim 13, wherein the distal end portion module and/or the proximal end portion module and/or the line body module comprise/comprises a multiplicity of sub-modules.

15. The set of electrode line modules according to claim 13, which comprises a plurality of line body modules of different lengths and/or proximal end portion modules which can be connected alternatively to the line body module and which have different device connection contacts and/or a plurality of distal end portion modules which can be connected alternatively to the line body module and which have different embodiments of electrode poles and/or which have a different mechanical/geometrical design.
